(19) United States
(12) Patent Application Publication
(10) Pub. No.: US 2024/0342362 A1
(43) Pub. Date: Oct. 17, 2024

(54) INFUSION PUMP THAT SETS PUMP PARAMETERS BASED ON CASSETTE TYPE

(71) Applicant: B. Braun Medical Inc., Bethlehem, PA (US)

(72) Inventors: Edwin Burnard, Schneckville, PA (US); Hans Schmolk, Perkasie, PA (US); Michael William Huk, Lehighton, PA (US); Michael Robert Pavlovic, Stroudsburg, PA (US); Vladislav Los, Palmerton, PA (US); Elisha James Endres, Bethlehem, PA (US)

(73) Assignee: B. Braun Medical Inc., Bethlehem, PA (US)

(21) Appl. No.: 18/134,650

(22) Filed: Apr. 14, 2023

(51) Int. Cl. A61M 5/142

(52) U.S. Cl. CPC A61M 5/142; A61M 2005/14208; A61M 2205/12; A61M 2205/60

(57) ABSTRACT

Techniques are provided for identifying an infusion cassette of an infusion pump that are simple to implement yet sophisticated enough to convey pump parameter data to the infusion pump when the infusion cassette is inserted into a receptacle of the infusion pump. The infusion pump includes a pump that acts on tubing to pump fluid through the tubing and a receptacle configured to receive an infusion cassette. The tubing passes through the infusion cassette and engages the pump when the infusion cassette is fully inserted. A detection system identifies the type of infusion cassette and sets and/or limits infusion pump parameters based on the detected type of infusion cassette. The detection system includes sensors within the infusion pump that detect the presence/absence of holes or other types of markings (e.g., colored regions or textured areas) on the infusion cassette in order to determine the type of infusion cassette and associated pumping parameters.

### FIELD OF THE INVENTION

The present disclosure is related to infusion pumps and, more particularly, to infusion pump cassette identification using a detection system including sensors that detect presence/absence of holes in or markings on the cassette body and to set and/or limit parameters using the identified cassette type (e.g., intravascular, neural/neuraxial, enteral, etc.).

### BACKGROUND

Infusion pumps deliver controlled doses of fluids such as medications, analgesics, and nutrition to patients. Infusion pumps are particularly well suited to delivering controlled doses of fluids over long periods of time, e.g., several hours or days. While many infusion pumps are designed for bedside use, there are ambulatory versions available. Ambulatory infusion pumps allow a patient to move around while the infusion pump is in use. These ambulatory pumps are also used in acute clinical settings to physically differentiate routes of therapy (e.g., intravascular, neural/neuraxial, enteral, etc.) so as to reduce possibility of wrong route medication errors.

Syringe pumps and peristaltic pumps are two conventional types of infusion pumps. A syringe pump depresses a cylinder within a syringe to deliver fluid from the syringe to a patient. A peristaltic pump acts on a tube to control the rate of fluid flow through the tube from a bottle or bag of fluid to a patient. Precise delivery of fluids is desirable to optimize treatment of a patient as there are many fluids where small variations can be critical.

Modular, multi-specialty fluid pumps have been described having a universal pump console that accepts removable pump cassettes (e.g., pump to infusion set interface element) with tube sets designed for various specialties that are attached to the universal pump console. The pump cassette that is attached to the pump console determines the categorical type of fluid pump mechanism. For example, U.S. 2021/0369940 describes a pump system including a pump console with a motor, a sensor, and a removable pump cassette attached to the pump console. The pump cassette includes a cassette identifier (e.g., a magnet or an RFID tag) that is detected by the sensor. The cassette identifier may dictate parameters such as motor operating parameters (e.g., default motor speed, timing, motor direction, etc.).

### SUMMARY

Alternative configurations for identifying an infusion cassette of an infusion pump are provided that are simple to implement yet sophisticated enough to convey pump parameter data to the infusion pump when inserted into a receptacle of the infusion pump. In sample configurations, the infusion pump includes a pump that acts on tubing to pump fluid through the tubing. The infusion pump has a receptacle configured to receive an infusion cassette. The tubing passes through the infusion cassette, which is inserted into the infusion pump such that the tubing engages the pump. The infusion pump implements a detection system to identify the type of infusion cassette and sets infusion pump parameters based on the detected type of infusion cassette. The detection system includes sensors within the pump that detect the presence/absence of holes or other types of markings (e.g., colored regions or textured areas) on the infusion cassette in order to determine the type of infusion cassette and associated pumping parameters.

In sample configurations, the detection system includes one or more sensors positioned within the receptacle of the infusion pump to detect holes, markings, or other properties of the infusion cassette to identify the type of infusion cassette. The sensors may be light sensors (e.g., optical, infrared (IR), etc.) that detect the presence/absence of holes or other markings or properties, a light sensor that detects the color of the cassette, mechanical sensors that detect indentations on the cassette, electrical sensors, electro-mechanical sensors, or other types of sensors that may be used to detect markings and properties so as to enable the infusion pump to distinguish one type of infusion cassette from another. The infusion sets thereby can be differentiated by including infusion cassettes specific to each infusion set's intended use and other features (e.g., inclusion of filters, access devices, tubing types, etc.). The ability to distinguish between infusion sets allows the pump to more safely infuse under a spectrum of intended uses by ensuring specific or limiting programmable infusion parameters.

The infusion cassette includes a cassette body having an indicator that can be sensed by the infusion pump detection system to determine the type of infusion cassette that has been inserted into the receptacle of the infusion pump. In one example, the infusion cassette has a wall with a detection surface area corresponding to sensors (e.g., IR sensors) within recesses of the infusion pump when the infusion cassette is fully inserted into the receptacle of the infusion pump. The detection surface area may include one or more through holes or other markings (e.g., colors) to convey data to the detection system.

The detection system includes an emitter/detector pair (with the detector adjacent or opposite the emitter) in the receptacle of the infusion pump corresponding to each through hole or marking and a controller configured to detect the presence/absence of through holes or markings at each of the through hole or marker positions and to determine the type of infusion cassette based on the detected presence/absence of through holes or markings. The marking on the detection surface of the infusion cassette may further include at least one colored region having a color representative of the code. The controller identifies the type of infusion cassette (e.g., intravascular, neural/neuraxial, enteral, etc.) by “reading” the through holes or markings of the infusion cassette. After identifying the type of infusion cassette, the infusion pump may set or limit pump parameters (e.g., delivery parameters, available drug options, etc.) other than the type of infusion cassette or infusion pump that is associated with that type of infusion cassette.

In sample configurations, an infusion pump is described that includes a housing having a pump and a receptacle, an infusion cassette that includes tubing for passage of an infusate, and an infusion cassette detection system. The infusion cassette fits into the receptacle such that the tubing is adjacent the pump when fully inserted. The infusion cassette further includes at least one hole or marking on a detection surface thereof that identifies at least the type of infusion cassette. The infusion cassette detection system detects a type of the infusion cassette when the infusion cassette is fully inserted in the receptacle of the housing. The detection system includes at least one emitter/detector pair including an emitter that emits light to the detection surface of the infusion cassette and a corresponding detector that receives reflected light, and a controller that determines a presence or absence of the at least one hole or marking on the detection surface from the reflected light and that determines the type of infusion cassette in accordance with the determination of the presence or absence of the at least one hole or marking. The controller, through engagement with on-board databases (e.g., drug delivery database), controls operation of the pump in accordance with the determined type of infusion cassette.

In the sample configurations, the controller determines the type of infusion cassette by determining a code represented by a presence or absence of the at least one hole or marking on the detection surface of the infusion cassette and uses the code as a search criterion into at least one of a drug library database or a library of available infusion cassettes. In addition to the type of infusion cassette, the code may represent at least one pumping parameter of the pump that the controller uses to control operation of the pump. The controller also may use the code to limit programming choices for the pump.

In addition, the controller may use the code to identify a mismatch between a selected drug and an installed infusion cassette and notify a user via a user interface of the infusion pump. For example, information provided to the user of the pump may serve as a safety measure (e.g., to prevent wrong-route medication errors, etc.). In the event this occurs, the pump controller may guide the user via the user interface to make informed pump parameter elections; those elections are then historically recorded.

The following description also describes a method of operating an infusion pump and a non-transitory controller-readable medium including instructions that when executed by one or more processors implement the steps of detecting the insertion of an infusion cassette into a receptacle of a housing of the infusion pump where the infusion cassette includes tubing adapted for passage of an infusate that is adjacent a pump of the housing when fully inserted in the receptacle; detecting at least one hole or marking on a detection surface of the infusion cassette that identifies at least the type of infusion cassette by using at least one emitter/detector pair to emit light to the detection surface of the infusion cassette and to receive reflected light indicative of a presence or absence of the at least one hole or marking on the detection surface; and determining a code represented by a presence or absence of the at least one hole or marking on the detection surface from the reflected light, the code representing the type of infusion cassette. The method also includes controlling operation of the pump in accordance with the determined type of infusion cassette.

In sample configurations, the method further includes using the code as a search criterion into at least one of a drug library database or a library of available infusion cassettes. Detecting the marking on the detection surface of the infusion cassette may further include detecting at least one colored region having a color representative of the code. Also, the code may represent at least one pumping parameter of the pump, and the method further includes controlling operation of the pump in accordance with the at least one pumping parameter. The code may represent at least one pumping parameter of the pump and may be used to control operation of the pump, limit programming choices for the pump, and/or to identify a mismatch between a selected drug and an installed infusion cassette and notify a user via a user interface.

### BRIEF DESCRIPTION OF THE DRAWINGS

The drawing figures depict multiple views of one or more implementations, by way of example only, not by way of limitations. In the figures, like reference numerals refer to the same or similar elements. The same numeral is used to represent the same or similar element across the multiple views. If multiple elements of the same or similar type are present, a letter may be used to distinguish between the multiple elements. When the multiple elements are referred to collectively or a non-specific one of the multiple elements is being referenced, the letter designation may be dropped.

FIG. 1 is a perspective view of an example ambulatory peristaltic infusion pump.

FIG. 2 is a perspective view of an example infusion cassette with a free flow prevention clamp for use with the ambulatory peristaltic infusion pump of FIG. 1.

FIG. 3 is a partial perspective view of the ambulatory peristaltic infusion pump of FIG. 1.

FIGS. 4 and 5 are cutaway views of the ambulatory peristaltic infusion pump of FIG. 1 illustrating pump sliders and cam rods for moving the pump sliders.

FIG. 6A is a side view of the ambulatory peristaltic infusion pump of FIG. 1 with the infusion cassette removed to show the emitter/detector pairs of the cassette type detection system in a sample configuration.

FIG. 6B is a diagram illustrating the emitter/detector pairs and the controller of the cassette type detection system in a sample configuration.

FIG. 7A is a diagram illustrating a side view of an infusion cassette as modified to include a detection surface area including a single through hole in a sample configuration.

FIG. 7B is a diagram illustrating a bottom view of the infusion cassette of FIG. 7A in a sample configuration.

FIG. 7C is a diagram illustrating a perspective view of the infusion cassette of FIG. 7A in a sample configuration.

FIG. 8A is a diagram illustrating a side view of an infusion cassette as modified to include a detection surface area including multiple through holes in a sample configuration.

FIG. 8B is a diagram illustrating a perspective view of the infusion cassette of FIG. 8A in a sample configuration.

FIG. 9 is a diagram illustrating a side view of an infusion cassette as modified to include a detection surface including markings (e.g., colored regions and/or textured areas) identifying the type of infusion cassette in a sample configuration.

FIG. 10 is a flow chart illustrating the operation of the pump controller in a sample configuration.

FIG. 11 is a functional block diagram illustrating a general-purpose computer hardware platform configured to implement the functional examples described with respect to FIGS. 1-10.

FIG. 12 is another functional block diagram illustrating a general-purpose computer hardware platform configured to implement the functional examples described with respect to FIGS. 1-10.

### DETAILED DESCRIPTION

In the following detailed description, numerous specific details are set forth by way of examples in order to provide a thorough understanding of the relevant teachings. However, it should be apparent to those skilled in the art that the present teachings may be practiced without such details. In other instances, well-known methods, procedures, components, and circuitry have been described at a relatively high-level, without detail, in order to avoid unnecessarily obscuring aspects of the present teachings. Moreover, while described with respect to an ambulatory peristaltic infusion pump for pain management, homecare, outpatient infusions, and the like, it will be appreciated by those skilled in the art that the pump cassette detection system described herein may be used with a variety of other pump types.

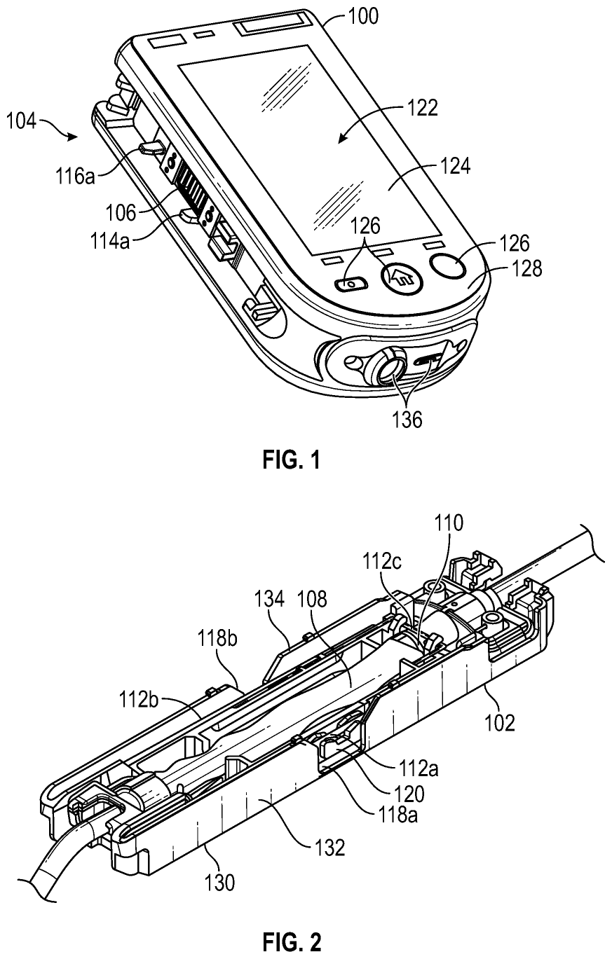

FIG. 1 depicts an example ambulatory peristaltic infusion pump 100, while FIG. 2 depicts an example infusion cassette 102 for use with the ambulatory peristaltic infusion pump 100. As illustrated in FIG. 1, the ambulatory peristaltic infusion pump 100 includes a receptacle 104 configured to receive the infusion cassette 102. During operation, the user inserts the infusion cassette 102 part way into the receptacle 104 of the ambulatory peristaltic infusion pump 100. The user closes a latch of the ambulatory peristaltic infusion pump 100, thus pulling the infusion cassette 102 fully flush into the receptacle 104. When software of the infusion pump 100 detects that the latch is closed (e.g., via a Hall effect sensor), the software checks the pressure of tubing of the installed infusion cassette 102 to ensure it is above a predetermined threshold. If the pressure sensors sense a sufficiently high pressure, then it is determined that an infusion cassette 102 is physically present. At this point, a cassette ID algorithm is invoked that uses the cassette ID sensors to read all the markings on the side of the cassette to then determine the cassette ID value. The cassette ID value is then passed along to user interface software of the pump to determine how to use the cassette ID for controlling pump parameters as described herein.

A peristaltic pump mechanism 106 within the receptacle 104 acts upon a tube 108 extending through a channel within the infusion cassette 102 to pump fluid from a fluid container (e.g., a bag or a bottle; not shown) into a patient. An example free flow prevention clamp 110 is positioned within the infusion cassette 102 to allow fluid flow through the tube 108 when the infusion cassette 102 is coupled to the ambulatory peristaltic infusion pump 100 within the receptacle 104 (during which time the peristaltic pump mechanism 106 controls fluid flow through the tube 108) and to selectively cut off fluid flow through the tube 108 when the infusion cassette 102 is not coupled to the ambulatory peristaltic infusion pump 100 in order to prevent unintentional fluid flow through the tube 108 (e.g., free flow).

The ambulatory peristaltic infusion pump 100 includes a user interface 122 for interacting with the ambulatory peristaltic infusion pump 100. The illustrated user interface 122 includes a display 124 (which may be a touchscreen) and buttons 126. A user controls the operation of the ambulatory peristaltic infusion pump 100 via the user interface 122. The ambulatory peristaltic infusion pump 100 additionally includes a housing 128 for containing and supporting the components of the ambulatory peristaltic infusion pump 100 such as the peristaltic pump mechanism 106, electronics, power supplies, and the like.

The free flow prevention clamp 110 includes a first elongate section 112a, a second elongate section 112b, and a clamping section 112c. The housing 130 of the infusion cassette 102 supports the free flow prevention clamp 110. The clamping section 112c is positioned within the cassette geometry such that, when the infusion cassette 102 is received within the receptacle 104 of the ambulatory peristaltic infusion pump 100, the clamping section 112c extends across the channel receiving the tube 108. The housing 130 of the infusion cassette 102 may be rigid plastic or other material capable of supporting the tube 108 and free flow prevention clamp 110.

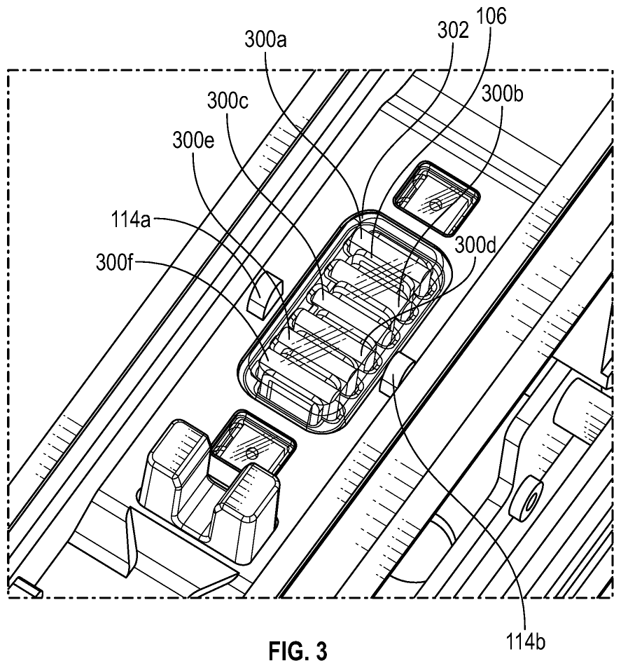

The ambulatory peristaltic infusion pump 100 also includes a pair of arc cams 114a and 114b (FIG. 3). First arc cam 114a is shown on one side of the receptacle illustrated in FIG. 1, but the second arc cam 114b is hidden from view. The pair of arc cams 114a and 114b engage the elongate sections 112a, 112b of the free flow prevention clamp 110 in order to lift the clamping section 112c. Additionally, the ambulatory peristaltic infusion pump 100 includes a pair of wedge cams 116a and 116b. A first wedge cam 116a is shown on one side of the receptacle 104 illustrated in FIG. 1, but the second wedge cam 116b is hidden from view. The pair of wedge cams 116a and 116b transition the free flow prevention clamp 110 from an open, manufactured/shipped state to an operational state, which is described in further detail below.

As shown in FIG. 2, the infusion cassette 102 also includes a first cutout 118a in a sidewall 132 of the infusion cassette 102 and a second cutout 118b in an opposite sidewall 134 of the infusion cassette 102. Additionally, the infusion cassette 102 includes a bypass button/pad 120 positioned on the first elongate section 112a adjacent a mid-point of the first elongate section 112a and the first cutout 118a. The bypass button/pad 120 and cutout 118a together facilitate engagement of the first elongate section 112a by a finger of an operator in order to manually lift the clamping section 112c to allow fluid flow through the tube 108 (e.g., for priming the infusion cassette 102) when the infusion cassette 102 is not received within the receptacle 104 of the ambulatory peristaltic infusion pump 100. The bypass button/pad 120 may be a press fit piece of rigid plastic. Although the bypass button/pad 120 is illustrated as only on the first elongate section 112a, the bypass button/pad 120 also may be provided on the second elongate section 112b.

The ambulatory infusion pump 100 further includes connector ports 136 that provide electronic access for control and for powering the ambulatory peristaltic infusion pump 100 when used in the configurations described below.

FIG. 3 depicts the peristaltic pump mechanism 106 of the ambulatory peristaltic infusion pump 100. The peristaltic pump mechanism 106 includes multiple pump sliders 300 (six pump sliders 300a-f illustrated in FIG. 3). A flexible barrier (seal) 302 separates the pump sliders 300 (and other pump components of a pumping mechanism) from the receptacle 104 receiving the infusion cassette 102 with the tube 108. The flexible barrier 302 provides a barrier between the fluid delivery apparatus/cassette and the pumping mechanism to prevent errant/leaked fluid from damaging internal components of the pumping mechanism.

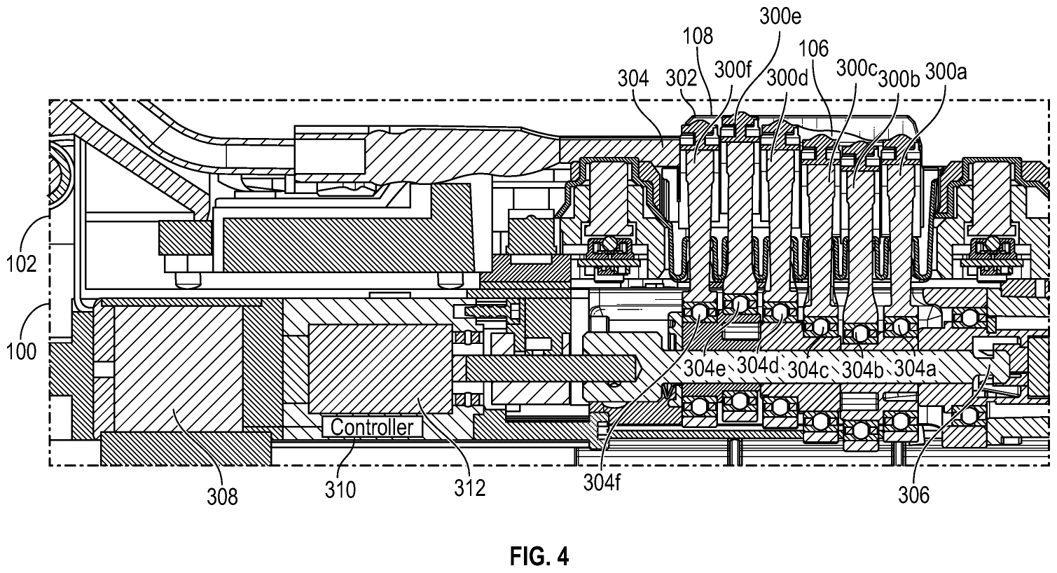

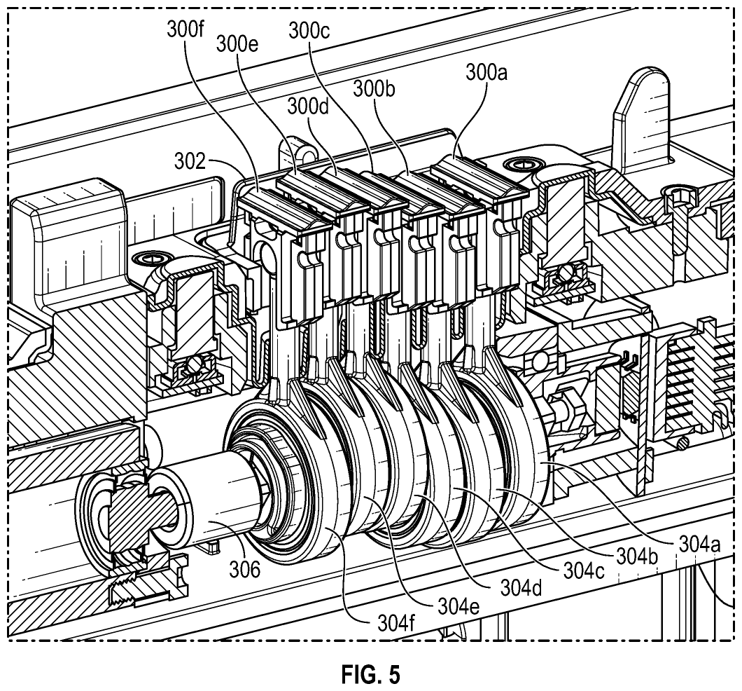

FIGS. 4 and 5 are cutaway views of the ambulatory peristaltic infusion pump 100 with the infusion cassette 102 inserted into the receptacle 104 of the ambulatory peristaltic infusion pump 100. Multiple cams 304 (six cams 304a-f) supported by a camshaft 306 of the ambulatory peristaltic infusion pump 100 act on respective pump sliders 300a-300f. The cams 304a-304f respectively raise and lower the pump sliders 300a-300f, which engage the tube 108 of the infusion cassette 102 in order to force fluid though the tube 108. A pump motor 308 under control of a controller 310 turns the camshaft 306 by way of a gearbox 312. As the camshaft 306 turns, the cams 304a-300f, which are offset from each other in an axial direction, raise and lower respective pump sliders 300a-300f. For example, cam 304a raises and lowers pump slider 300a; cam 304b raises and lowers pump slider 300b, and the like. The controller 310 may be a standalone or embedded processor configured to carry out instructions in order to control operations of the ambulatory peristaltic infusion pump 100.

The controller 310 may include a main controller such as a dual core 32 bit processor from NXP of Eindhoven, Netherlands (e.g., model #MCIMX7S5EVM08SC), a microcontroller from NXP (e.g., model #MKV31F512VLH12), a pump motor driver from ST Microelectronics of Geneva, Switzerland (e.g., model #STSPIN250), and a magnetic encoder from Austriamicrosystems of Premstaetten, Austria (e.g., model number AS5601-ASOM). The microcontroller receives pump camshaft revolutions per minute (RPM) corresponding to the infusion flow rate from a system control core of the main processor. The microcontroller develops a pulse width modulation (PWM) motor drive parameter relating to the desired camshaft RPM. The PWM output of the microcontroller becomes the motor drive input to the pump motor driver, which contains motor drive transistors and protection circuitry. The rotation of the camshaft 306 of the pumping mechanism is measured by the magnetic encoder. At specified time intervals, the output of the encoder is read by the microcontroller, which uses the encoder value to compute the speed of the camshaft 306 and the position of the pump rotation. These values are then used to modify the PWM output to maintain the correct camshaft RPM.

An ambulatory peristaltic infusion pump 100 as described herein thus may include a linear peristaltic pump mechanism 106 that acts on tubing 108 to pump fluid through the tubing 108. The tubing 108 passes through the infusion cassette 102, which is inserted into the ambulatory peristaltic infusion pump 100 such that the tubing 108 engages the linear peristaltic pump mechanism 106. The ambulatory peristaltic infusion pump 100 may be configured based on the type of infusion cassette 102.

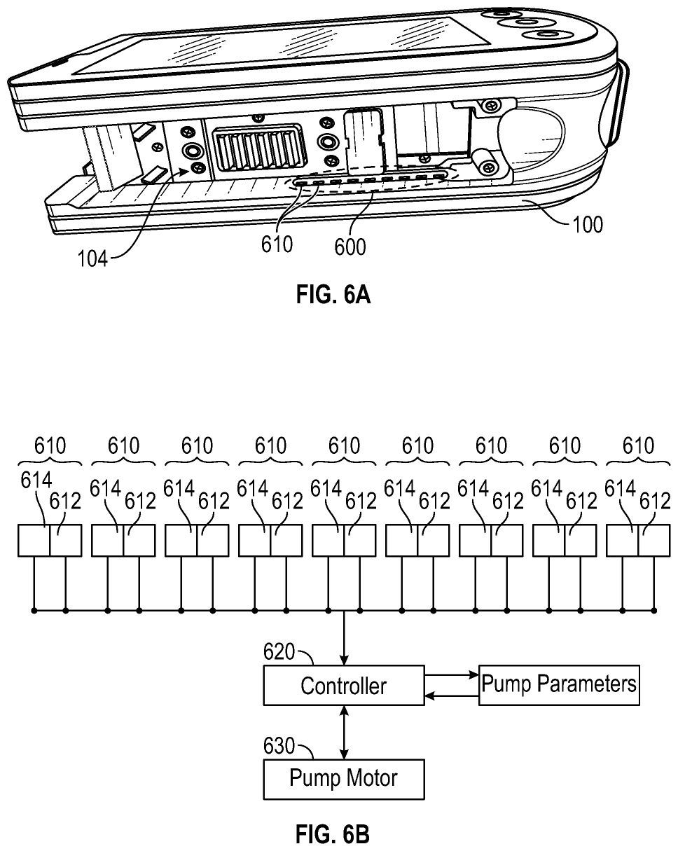

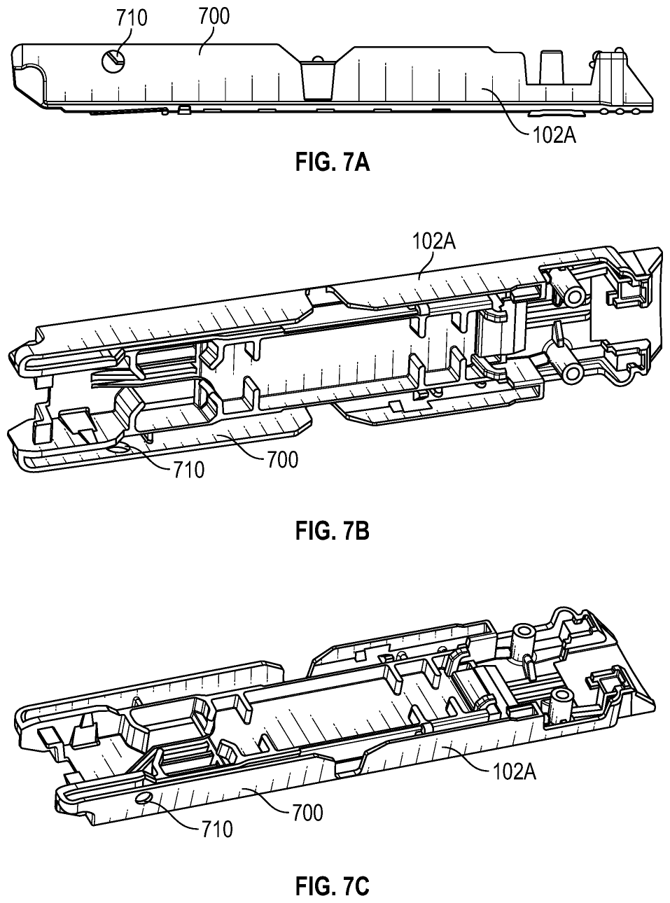

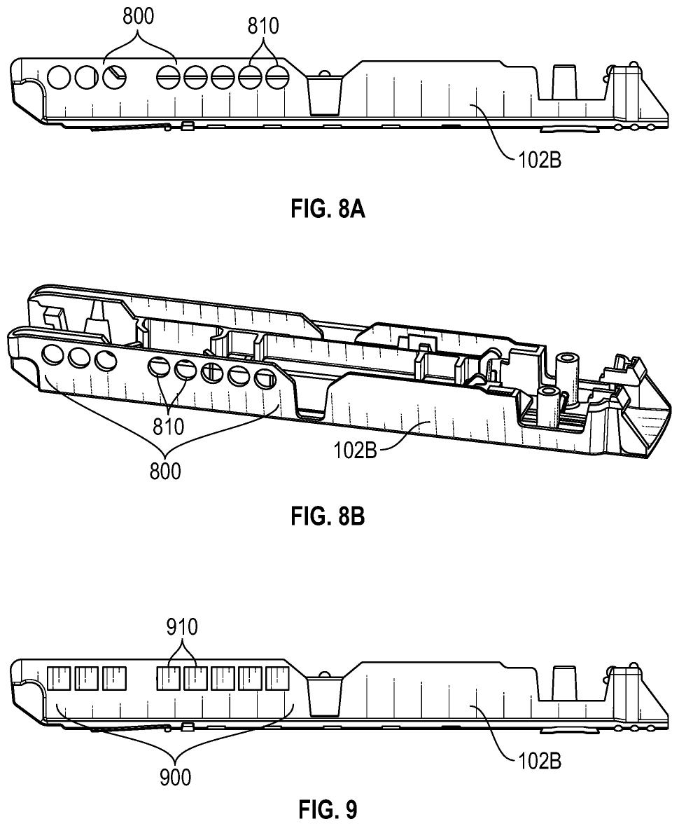

FIG. 6A is a side view of the ambulatory peristaltic infusion pump 100 of FIG. 1 with the infusion cassette 102 removed to show a detection region 600 including emitter/detector pairs 610 of a sensor for detecting the cassette type in a sample configuration. The emitter/detector pairs 610 detect the presence/absence of holes and/or markings in the side of the infusion cassette 102. The infusion cassette 102 may include a single hole (FIGS. 7A-7C, element 710) or a plurality of holes (FIGS. 8A-8B, element 810) or other types of surface markings such as colored regions (FIG. 9, element 910) that represent the type (and optionally pumping parameters) of the infusion cassette 102. The emitter/detector pairs 610 or other sensors may be used to detect other features of the infusion cassette 102, such as the shape of the infusion cassette 102 including surface textures, ribs, or other features intended to reflect incoming light in non-normal directions, the presence/absence of notches, the presence/absence of unique markings such as quick response (QR) codes, radiofrequency identification (RFID) tags, product name, and the like.

As noted above, the ambulatory peristaltic infusion pump 100 has a receptacle 104 configured to receive an infusion cassette 102. The emitter/detector pairs 610 of the detection system are positioned within the receptacle 104 as illustrated in FIG. 6A to detect the presence/absences of holes or other markings of the infusion cassette 102 to identify the type of infusion cassette 102. The emitter/detector pairs 610 may be optical sensors that detect the presence/absence of holes or other markings or properties of the infusion cassette 102, an optical sensor that detects the color of the infusion cassette 102, mechanical sensors that detect indentations on the infusion cassette 102, electrical sensors, electro-mechanical sensors, or other combinations of sensors and markings/properties known to those skilled in the art that are used to enable the ambulatory peristaltic infusion pump 100 to distinguish one type of infusion cassette 102 from another.

FIG. 6B is a diagram illustrating the emitter/detector pairs 610 and the controller 620 of the cassette type detection system in a sample configuration. As illustrated, each emitter/detector pair 610 includes an emitter 612 and a detector 614. The emitter/detector pairs 610 are controlled by a controller 620 that instructs the emitters 612 to emit light (e.g., IR light) signals and receives indications from each detector 614 indicating whether the light was reflected by the side of the infusion cassette 102 or passed through one or more holes of the infusion cassette 102 to the inside of the infusion cassette 102. Conversely, the emitter/detector pairs 610 may detect the color and/or contours of markings on the infusion cassette 102. The respective holes or other markings convey information and/or signals through the emitter/detector pairs 610 as to the type of infusion cassette 102 has been inserted. The signals from each of the detectors 614 are provided to the controller 620 for a determination of what type of infusion cassette 102 has the detected pattern or holes and/or markings. The controller 620 determines which pump parameters to apply per the associated detected type of infusion cassette 102. The controller 620, per the pump parameters associated with the type of infusion cassette 102, controls the pump motor 630 accordingly to drive the operation of the ambulatory peristaltic infusion pump 100. Constraints/limits on pump programming may be displayed on and engaged by the user on the pump's user interface 122.

In sample configurations, the infusion cassette 102 includes a housing 130 having an indicator that can be sensed by the infusion pump detection system to determine pump type. In a first example shown in FIGS. 7A-7C, the infusion cassette 102A has a wall with a detection surface area 700 that is adjacent the detection region 600 of the ambulatory peristaltic infusion pump 100 when the infusion cassette 102 has been fully inserted into the receptacle 104 and secured into place. Once the infusion cassette 102 has been inserted into the receptacle 104 and secured into place, the respective emitters 612 of the detection region 600 are illuminated opposite the detection surface area 700. The signals coming from the respective detectors 614 are used by the controller 620 to detect the hole 710 of the detection surface area 700 of the infusion cassette 102. In sample configurations, the emitter/detector pairs 610 include infrared (IR) sensors 610 within the receptacle 104 of the ambulatory peristaltic infusion pump 100 that align with the hole 710 (or absence of a hole 710) when the infusion cassette 102 is fully inserted into the receptacle 104.

In the configuration of FIGS. 7A-7C where the infusion cassette 102A includes a detection surface area 700 including a single through hole 710, the infusion cassette 102A may convey only 1 bit of data. In such case, the through hole 710 may convey the presence/absence of the infusion cassette 102 or convey whether the infusion cassette 102 is one of two types (cassette type with hole 710 or cassette type without hole 710). Conversely, the detection surface area 700 in the configuration of FIGS. 7A-7C may provide access by the emitter/detector pairs 610 to a unique marking that uniquely identifies the type of infusion cassette 102. For example, the unique marking may be a texture or otherwise non-planar surface, an icon, a QR code, a bar code, a watermark, or other type of marking that is unique to the infusion cassette 102. The output of the single emitter/detector pair 610 is provided to the controller 620 for identification of the type of cassette 102 from the presence/absence of the hole 710 or for identification of the unique marking.

In the configuration of FIGS. 8A-8B, the infusion cassette 102B includes a detection surface area 800 including multiple through holes 810. In this configuration, the infusion cassette 102B may convey multiple bits of data. In the illustrated configuration, up to 9 holes may be provided, which conveys up to 9 bits of data resulting in up to 2⁹=512 hole/no-hole variations. Such data may be mapped to up to 512 different infusion cassette and pumping parameter configurations stored in a memory of the ambulatory peristaltic infusion pump 100. As in the configuration of FIGS. 7A-7C, the detection surface area 800 in the configuration of FIGS. 8A-8B also may provide access by the emitter/detector pairs 610 to a unique marking that uniquely identifies the type of infusion cassette 102. For example, the unique marking may be a texture or otherwise non-planar surface, an icon, a QR code, a bar code, a watermark, or other type of marking that is unique to the infusion cassette 102. The outputs of the emitter/detector pairs 610 are provided to the controller 620 for identification of the type of cassette 102 from the presence/absence of the holes 810 and for identification of the unique marking.

In sample configurations, a code represented by the presence/absence of the holes and/or the unique markings is captured by the detection system to identify the type of infusion cassette 102 (e.g., intravascular, neural/neuraxial, enteral, etc.) by “reading” the indicator(s) (e.g., holes) or markings 710 or 810 on or associated with the infusion cassette 102. After identifying the type of infusion cassette 102, the ambulatory peristaltic infusion 100 pump may set pump parameters (e.g., delivery thresholds, available drug options, etc.) that are associated with that type of infusion cassette 102. Such parameters may be stored in a memory of the peristaltic infusion pump 100 and accessed based on the read holes or markings 710 or 810.

A code represented by the holes or markings 710 or 810 may further provide additional programming safety by constraining programming choices available through interaction and limitations of a drug or care area within a drug library database of the ambulatory peristaltic infusion pump 100. This feature assists in preventing wrong route medication errors (i.e., medications intended for a specific region of the body are prevented from being infused into the wrong region of the body). For example, a code represented by the holes or markings 710 or 810 would limit the programming choices for an Epidural Infusion Set to only specific epidural drugs within the drug library, or pop up a notice to a user in the event of a mismatch between the selected drug and the installed infusion cassette 102. In this example, the code read from the infusion cassette 102 does not directly influence, change, or set the pumping parameters (e.g., flow rate, pressure settings, etc.) but does warn the user when the user sets unexpected or incompatible pumping parameters.

FIG. 9 is a diagram illustrating a side view of an infusion cassette 102B modified to include a detection surface 900 including markings (e.g., colored regions) 910 identifying the type of infusion cassette 102. In this configuration, each marking 910 may have a color that is read by the emitter/detector pairs 610 to identify unique code combinations representative of the type of infusion cassette 102 as well as pump parameters of the ambulatory peristaltic infusion pump 100. For example, a red marking could represent the value “0,” a blue marking could represent the value “1,” a green marking could represent the value “3,” and the like.

To set up the ambulatory peristaltic infusion pump 100 for operation, an infusion cassette 102 is inserted into the receptacle 104. The detection system includes a series of emitter/detector pairs 610 positioned within the receptacle 104. The respective emitters 612 of the emitter/detector pairs 610 emit light towards the detection surface 700/800/900 of the fully inserted infusion cassette 102. As described above, the detection surface 700/800 includes holes 710 or 810 that extend through the detection surface 700/800 of the body of the infusion cassette 102. The emitted light is either reflected by the detection surface 700/800 in the absence of a hole 710/810 or passes through the hole 710/810 and is either not reflected back or is reflected back at a different timing and/or intensity. The light reflection characteristics detected by the detector(s) 614 differ between non-reflected light and light reflected at a different timing and/or intensity, and these light reflection characteristics are translated into values understood by the controller 620. The respective values are provided to the controller 620 for identification of the code represented by the hole(s) 710/810. Similarly, the respective emitters 612 may emit light toward the detection surface 900 including the colored markings 910 of the configuration of FIG. 9. In this case, the detector(s) 614 may detect the color of the markings 910, which is provided to the controller 620 for interpretation. For example, the detector may detect different reflections for each color through use of, for example, coatings or infrared absorbing/intensifying inks to print the marks.

In one example, a series of nine sensors consisting of 940 nm wavelength IR light emitter-detector pairs 610 are positioned within the receptacle 104 of the ambulatory peristaltic infusion pump 100. The presence/absence of corresponding holes 710/810 in the infusion cassette 102 effectively creates a code that can be used to identify the type of infusion cassette 102 inserted into the ambulatory peristaltic infusion pump 100.

In sample configurations, two of the nine sensors of the light emitter-detector pairs 610 may be used to calibrate the detection system. One of the sensors may take measurements that correspond to a hole 710/810 in the detection surface 700/800 of the infusion cassette 102 when the infusion cassette 102 is inserted and another sensor may take measurements that correspond to the absence of a hole 710/810 in the detection surface 700/800 of the infusion cassette 102 when the infusion cassette 102 is inserted. This calibration process is useful for detecting the presence/absence of holes by the other sensors even if the infusion cassettes 102 may have detection surfaces in different colors. After calibration, the remaining sensors may be used to determine properties of the infusion cassette 102 based on the presence/absence of holes (e.g., intravascular, neural/neuraxial, enteral, etc.).

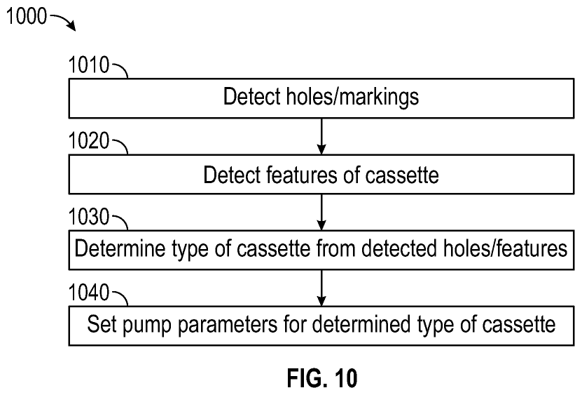

FIG. 10 is a flow chart illustrating the process 1000 followed by the controller 620 during operation in a sample configuration. As illustrated, the controller 620 instructs the emitters 612 to emit light (e.g., infrared light) signals and then receives the outputs of the detectors 614 representative of the hole configurations and/or markings on the infusion cassette 102 at 1010. The controller 620 may also receive at 1020 data representative of other features of the infusion cassette 102, such as the shape of the infusion cassette 102, the presence/absence of notches, the presence/absence of unique markings such as a texture or otherwise non-planar surface, an icon, QR codes, RFID tags, product name, and the like, as detected by other sensors on the ambulatory peristaltic infusion pump 100. At 1030, the controller 620 determines the type of cassette from the data received at 1010 and 1020. For example, the binary code provided by the detectors 614 may be used as a search criterion into a drug library database and/or a library of available infusion cassettes 102. Optionally, the controller 620 may read pump parameter values and/or drug infusion delivery parameters from the drug library database and/or the library of available infusion cassettes 102. The controller 620, per the pump parameters associated with the type of infusion cassette 102, controls the pump motor 630 accordingly to drive the operation of the ambulatory peristaltic infusion pump 100 at 1040.

It will be appreciated that fewer or more holes 710/810 and markings 910 may be used than are shown in the figures. For example, the holes 710/810 need not be cylindrical in shape but may be prismatic holes of any configuration such that the emitter 612 and the receiver 614 pairs can detect a difference in reflected light. The number of holes 710/810 and markings 910 is dependent upon the amount of data to be conveyed (e.g., the number of infusion cassette types and drug delivery configurations). It will also be appreciated that the holes 710/810 and markings 910 need not be used to adjust any direct pumping parameters such as flow rate, pressure settings, accuracy, etc. but instead may be limited to identification of the type of infusion cassette 102 and any unique characteristics of the infusion cassette 102. In such a configuration, the pumping parameters may be set during a programming process for programming the ambulatory peristaltic infusion pump 100.

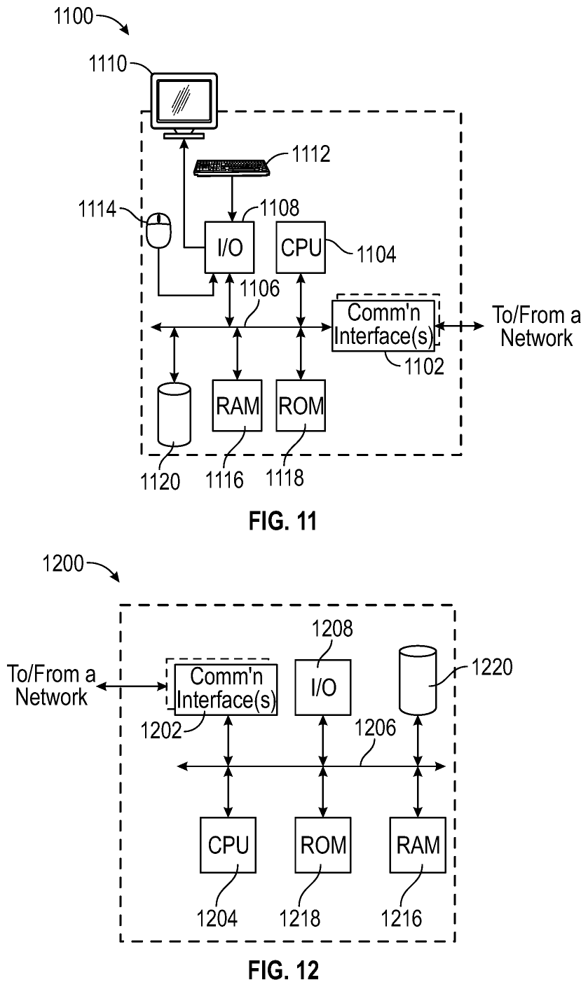

FIGS. 11 and 12 are functional block diagrams illustrating general-purpose computer hardware platforms configured to implement the functional examples described above with respect to FIGS. 1-10.

Specifically, FIG. 11 illustrates an example computer platform 1100 and FIG. 12 depicts an example computer 1200 with user interface elements, as may be used to implement in a personal computer, ambulatory peristaltic infusion pump 100, or other type of workstation or terminal device. It is believed that those skilled in the art are familiar with the structure, programming and general operation of such computer equipment and as a result the drawings should be self-explanatory.

Hardware of an example computer 1100 (FIG. 11) includes a data communication interface 1102 for packet data communication. The computer 1100 also includes a central processing unit (CPU) 1104, in the form of circuitry forming one or more processors, for executing program instructions. The computer platform hardware typically includes an internal communication bus 1106, program and/or data storage 1116, 1118, and 1120 for various programs and data files to be processed and/or communicated by the computer 1100, although the computer 1100 often receives programming and data via network communications. In one example, as shown in FIG. 11, the computer 1100 includes a video display unit 1110, (e.g., a liquid crystal display (LCD) or a cathode ray tube (CRT)), an alphanumeric input device 1112 (e.g., a keyboard), and a cursor control device 1114 (e.g., a mouse), each of which communicate via an input/output device (I/O) 1108. The hardware elements, operating systems and programming languages of such computers 1100 are conventional in nature, and it is presumed that those skilled in the art are adequately familiar therewith. Of course, the computer functions may be implemented in a distributed fashion on a number of similar hardware platforms, to distribute the processing load.

Hardware of a user terminal device 1200, such as a PC or tablet computer, similarly includes a data communication interface 1202, CPU 1204, main memory 1216 and 1218, one or more mass storage devices 1220 for storing user data and the various executable programs, an internal communication bus 1206, and an input/output device (I/O) 1208 (see FIG. 12).

Aspects of the methods for pump control, as outlined above, may be embodied in programming in general purpose computer hardware platforms (such as described above with respect to FIGS. 11 and 12), e.g., in the form of software, firmware, or microcode executable by a networked computer system such as a server or gateway, and/or a programmable nodal device. Program aspects of the technology may be thought of as “products” or “articles of manufacture” typically in the form of executable code and/or associated data that is carried on or embodied in a type of machine readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software, from one computer or processor into another, for example, from a processor 1104 of the system 1100 and/or from a controller 310 or 620 of an ambulatory peristaltic infusion pump 100 to a computer or software of another system (not shown). Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to one or more of “non-transitory,” “tangible” or “storage” media, terms such as computer or machine “readable medium” refer to any medium that participates in providing instructions to a processor for execution.

Hence, a machine-readable medium may take many forms, including but not limited to, a tangible storage medium, a carrier wave medium or physical transmission medium. Non-transitory storage media include, for example, optical or magnetic disks, such as any of the storage devices in any computer(s) or the like. It may also include storage media such as dynamic memory, for example, the main memory of a machine or computer platform. Tangible transmission media include coaxial cables; copper wire and fiber optics, including the wires that include a bus within a computer system. Carrier-wave transmission media can take the form of electric or electromagnetic signals, or acoustic or light waves such as those generated during radio frequency (RF) and light-based data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media may be involved in carrying one or more sequences of one or more instructions to a processor for execution.

Program instructions may include a software or firmware implementation encoded in any desired language. Programming instructions, when embodied in machine readable medium accessible to a processor of a computer system or device, render computer system or device into a special-purpose machine that is customized to perform the operations specified in the program performed by the controller 310 or 620 of the ambulatory peristaltic infusion pump 100.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is ordinary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents.

Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” “includes,” “including,” or any other variation thereof, are intended to cover a non-exclusive inclusion, such that a process, method, article, or apparatus that includes a list of elements does not include only those elements but may include other elements not expressly listed or inherent to such process, method, article, or apparatus. An element preceded by “a” or “an” does not, without further constraints, preclude the existence of additional identical elements in the process, method, article, or apparatus that includes the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

In addition, in the foregoing Detailed Description, it can be seen that various features are grouped together in various examples for the purpose of streamlining the disclosure. This method of disclosure is not to be interpreted as reflecting an intention that the claimed examples require more features than are expressly recited in each claim. Rather, as the following claims reflect, the subject matter to be protected lies in less than all features of any single disclosed example. Thus, the following claims are hereby incorporated into the Detailed Description, with each claim standing on its own as a separately claimed subject matter.

While the foregoing describes what is considered to be the best mode and other examples, it is understood that various modifications may be made and that the subject matter disclosed herein may be implemented in various forms and examples, and that they may be applied in numerous applications, only some of which have been described herein. It is intended by the following claims to claim any and all modifications and variations that fall within the true scope of the present concepts.

What is claimed is:

1. An infusion pump, comprising:
a housing comprising a pump and a receptacle;
an infusion cassette that includes tubing for passage of an infusate, the infusion cassette fitting into the receptacle whereby the tubing is adjacent the pump when fully inserted, the infusion cassette further comprising at least one hole or marking on a detection surface thereof that identifies at least type of infusion cassette; and
an infusion cassette detection system that detects the type of the infusion cassette when the infusion cassette is fully inserted in the receptacle of the housing, the detection system comprising at least one emitter/detector pair including an emitter that emits light to the detection surface of the infusion cassette and a corresponding detector that receives reflected light, and a controller that determines a presence or absence of the at least one hole or marking on the detection surface from the reflected light and that determines the type of infusion cassette in accordance with the determination of the presence or absence of the at least one hole or marking.

2. The infusion pump of claim 1, wherein the controller controls operation of the pump in accordance with the determined type of infusion cassette.

3. The infusion pump of claim 1, wherein the controller determines the type of infusion cassette by determining a code represented by a presence or absence of the at least one hole or marking on the detection surface of the infusion cassette and uses the code as a search criterion into at least one of a drug library database or a library of available infusion cassettes.

4. The infusion pump of claim 3, wherein the marking on the detection surface of the infusion cassette comprises at least one colored region having a color representative of the code.

5. The infusion pump of claim 3, wherein the code represents at least one pumping parameter of the pump.

6. The infusion pump of claim 5, wherein the controller controls operation of the pump in accordance with the at least one pumping parameter.

7. The infusion pump of claim 3, wherein the controller uses the code to limit programming choices for the pump.

8. The infusion pump of claim 3, further comprising a user interface, wherein the controller uses the code to identify a mismatch between a selected drug and an installed infusion cassette and notifies a user via the user interface.

9. The infusion pump of claim 1, wherein the detection system further comprises at least one mechanical, electrical, or electro-mechanical sensor adapted to detect at least one of a shape of the infusion cassette, an indentation in the infusion cassette, a textured surface of the infusion cassette, a presence or absence of at least one notch in the infusion cassette, or a presence or absence of at least one of a quick response (QR) code, a radiofrequency identification (RFID) tag, or a product name.

10. The infusion pump of claim 1, further comprising two sensors that are adapted to calibrate the detection system, wherein a measurement by a first sensor may correspond to a hole or marking in the detection surface of the infusion cassette when the infusion cassette is fully inserted into the receptacle and a measurement by a second sensor may correspond to an absence of a hole or marking in the detection surface of the infusion cassette when the infusion cassette is fully inserted.

11. A method of operating an infusion pump, comprising:
inserting an infusion cassette into a receptacle of a housing of the infusion pump whereby tubing of the infusion pump adapted for passage of an infusate is adjacent a pump of the housing when fully inserted in the receptacle;
detecting at least one hole or marking on a detection surface of the infusion cassette that identifies at least type of infusion cassette by using at least one emitter/detector pair to emit light to the detection surface of the infusion cassette and to receive reflected light indicative of a presence or absence of the at least one hole or marking on the detection surface; and
determining a code represented by a presence or absence of the at least one hole or marking on the detection surface from the reflected light, the code representing the type of infusion cassette.

12. The method of claim 11, further comprising controlling operation of the pump in accordance with the determined type of infusion cassette.

13. The method of claim 11, further comprising using the code as a search criterion into at least one of a drug library database or a library of available infusion cassettes.

14. The method of claim 11, wherein detecting the marking on the detection surface of the infusion cassette comprises detecting at least one colored region having a color representative of the code.

15. The method of claim 11, wherein the code represents at least one pumping parameter of the pump, further comprising controlling operation of the pump in accordance with the at least one pumping parameter.

16. The method of claim 11, further comprising using the code to limit programming choices for the pump.

17. The method of claim 11, further comprising using the code to identify a mismatch between a selected drug and an installed infusion cassette and notifies a user via a user interface.

18. The method of claim 11, further comprising detecting at least one of a shape of the infusion cassette, an indentation in the infusion cassette, a textured surface of the infusion cassette, a presence or absence of at least one notch in the infusion cassette, or a presence or absence of at least one of a quick response (QR) code, a radiofrequency identification (RFID) tag, or a product name.

19. The method of claim 11, further comprising calibrating a detection system using a measurement by a first sensor to correspond to a hole or marking in the detection surface of the infusion cassette when the infusion cassette is fully inserted into the receptacle and a measurement by a second sensor to correspond to an absence of a hole or marking in the detection surface of the infusion cassette when the infusion cassette is fully inserted.

20. A non-transitory controller-readable storage medium storing controller-executable instructions that, when executed by a controller of an infusion pump, cause the infusion pump to perform operations comprising:
detecting insertion of an infusion cassette into a receptacle of a housing of the infusion pump whereby tubing of the infusion pump adapted for passage of an infusate is adjacent a pump of the housing when fully inserted in the receptacle;
detecting at least one hole or marking on a detection surface of the infusion cassette that identifies at least type of infusion cassette by using at least one emitter/detector pair to emit light to the detection surface of the infusion cassette and to receive reflected light indicative of a presence or absence of the at least one hole or marking on the detection surface; and
determining a code represented by a presence or absence of the at least one hole or marking on the detection surface from the reflected light, the code representing the type of infusion cassette.

21. The controller-readable storage medium of claim 20, further comprising instructions that when executed by the controller of the infusion pump cause the infusion pump to use the code to identify a mismatch between a selected drug and an installed infusion cassette and to notify a user via a user interface.

22. The controller-readable storage medium of claim 20, further comprising instructions that when executed by the controller of the infusion pump cause the infusion pump to detect at least one of a shape of the infusion cassette, an indentation in the infusion cassette, a textured surface of the infusion cassette, a presence or absence of at least one notch in the infusion cassette, or a presence or absence of at least one of a quick response (QR) code, a radiofrequency identification (RFID) tag, or a product name.

23. The controller-readable storage medium of claim 20, further comprising instructions that when executed by the controller of the infusion pump cause the infusion pump to calibrate a detection system using a measurement by a first sensor to correspond to a hole or marking in the detection surface of the infusion cassette when the infusion cassette is fully inserted into the receptacle and a measurement by a second sensor to correspond to an absence of a hole or marking in the detection surface of the infusion cassette when the infusion cassette is fully inserted.
